(19) United States
(12) Patent Application Publication
(10) Pub. No.: US 2013/0078317 A1
(43) Pub. Date: Mar. 28, 2013

(54) ZINC-CONTAINING COMPOSITIONS FOR THE TREATMENT OF DISEASES, ILLNESSES AND SYNDROMES ASSOCIATED WITH EXPOSURE TO PORE FORMING TOXINS

(71) Applicant: Angel Anne Yanagihara, Honolulu, HI (US)

(72) Inventor: Angel Anne Yanagihara, Honolulu, HI (US)

(21) Appl. No.: 13/498,116

(22) PCT Filed: Sep. 23, 2010
(86) PCT No.: PCT/US10/50061

§ 371 Date: Nov. 19, 2012

Related U.S. Application Data
(60) Provisional application No. 61/245,238, filed on Sep. 23, 2009.

(51) Int. Cl. A61K 33/30; A61K 31/7016

(52) U.S. Cl. CPC A61K 33/30; A61K 31/7016
USPC 424/642; 556/131; 514/53

(57) ABSTRACT

Embodiments of the invention relate to compositions and methods of using the same to treat conditions caused by exposure to a pore-forming toxin.

### STATEMENT REGARDING FEDERALLY-SPONSORED RESEARCH

This invention was supported in part with government support under Grant No. U54 NS039406, awarded by the National Institutes of Health; under Grant No. G12 RR003061-22, awarded by the National Institutes of Health; under Grant No. R21 DA024444-01A1, awarded by the National Institutes of Health; and under Grant No. P20 RR016453, awarded by the National Institutes of Health. The government has certain rights in the invention.

### FIELD OF THE INVENTION

The invention disclosed herein generally relates to compositions and methods of using the same to treat conditions caused by exposure to a pore-forming toxin.

### BACKGROUND

Each year hundreds of thousands of beachgoers are stung by cnidarian family members, which include the anthozoan (anemone, corals), hydrozoans, scyphozoans (common jellyfish), and cubozoans (tropical cuboidal jellies with venoms that result in high morbidity and mortality). For example, life-threatening Chironex fleckeri envenomations occur each year, from November to May, in North Queensland, Australia. There is no clearly effective specific therapy available; the current “anti-venom” is used without clinical validation and has been linked to negative outcomes. Current treatments are directed at relief of symptoms or, in serious cases, support of cardiovascular integrity after envenomation. A few currently available “Sting Relief” type sprays are typically comprised of ingredients such as vinegar, lidocaine, papain, aloe, eucalyptus oil, and menthol. Thus, there is a need for effective therapies to reduce the morbidity and mortality outcomes for cnidarian envenomations, which include addressing inflammation, pain, and systemic and cardiovascular outcomes related to cnidarians envenomations.

In addition, pore-forming toxins, or porins, represent an ancient and conserved toxic exudate of most pathogenic bacteria, including staphylococci, streptococci, anthrax, Clostridium, and E. coli, and are a major constituent of bee and certain spider venoms. Potent membrane disruptive porins allow the evasion of host phagocytosis in bacterial infection and rapid prey cytolysis in invertebrate envenomation. Thus, they constitute a fundamental mechanism for infection and prey capture. Thus; effective therapies for treatment of cnidarian envenomations will have general applicability to all conditions associated with pore-forming toxins.

### SUMMARY OF THE INVENTION

Embodiments of the invention include a method for treating a mammal suffering from a disease, illness, syndrome or condition resulting from the action of a pore-forming toxin, including administering to the mammal a therapeutically effective dosage of a zinc-containing compound. In some embodiments, the zinc-containing compound is administered intravenously. In some embodiments, the zinc compound is administered via transdermal patch.

In some embodiments, the zinc containing compound is zinc acetate. In some embodiments, the zinc containing compound is zinc malate. In some embodiments, the zinc containing compound is zinc chloride. In some embodiments, the zinc containing compound is zinc sulfate. In some embodiments, the zinc containing compound is zinc propionate. In some embodiments, the zinc containing compound is zinc butyrate. In some embodiments, the zinc containing compound is zinc oxalate. In some embodiments, the zinc containing compound is zinc malonate. In some embodiments, the zinc containing compound is zinc succinate. In some embodiments, the zinc containing compound is zinc gluconate.

In some embodiments, the disease or condition can be, for example, bacterial sepsis, Irukandji syndrome, cardiovascular collapse, pulseless electrical activity (PEA) hyperkalemia, hemolysis, cytokine and histamine release, catecholamine surge, and the like.

In some embodiments, the method additionally includes administering to the mammal a therapeutically effective dose of a composition including a carbohydrate. In some embodiments, the carbohydrate includes D-lactulose.

In embodiments of the invention, a method for treating a mammal suffering from a disease resulting from the action of a pore-forming toxin is provided, the method including administering to the mammal a therapeutically effective dosage of a composition including a carbohydrate. In some embodiments, the carbohydrate is D-lactulose.

Embodiments of the invention are also directed to the use of a zinc-containing composition for the manufacture of a medicament for treating a disease associated with a pore-forming toxin.

In embodiments of the invention, the use of a composition including a carbohydrate for the manufacture of a medicament for treating a disease associated with a pore-forming toxin is provided.

### BRIEF DESCRIPTION OF THE DRAWINGS

FIG. 1 is an XY-plot of the dose-response curve illustrating that zinc gluconate fully inhibits potassium efflux induced by Carybdea alata venom in isolated red blood cells (2% RBC).

FIG. 2 is an XY-plot of the dose-response curve showing that zinc gluconate fully inhibits potassium efflux induced by Chironex fleckeri venom in isolated red blood cells (2% RBC).

FIG. 3 is an XY-plot of the dose-response curve illustrating that zinc gluconate inhibits potassium efflux induced by Chironex fleckeri venom in whole blood.

FIG. 4 is an XY-plot of the dose-response curve showing that zinc gluconate inhibits potassium efflux induced by purified hemolysin from Carybdea alata in isolated red blood cells (2% RBC).

FIG. 5A is a plot chart illustrating that of zinc gluconate inhibits hemolysis induced by Carybdea alata venom exposure in isolated red blood cells (2% RBC). Data is provided as absorbance (at 405 nm wavelength) of plasma aliquots at specific timepoints.

FIG. 5B is a semi-log XY plot . . . of the data showing that increasing amounts of zinc gluconate delays the half-time of hemolysis occuring in isolated red blood cells (2% RBC) after exposure to Carybdea alata venom. Data is provided as absorbance (at 405 nm wavelength) of plasma aliquots taken at specific timepoints as a function of dose of Carybdea alata venom with different concentrations of zinc gluconate ranging from 0.62 mM to 5 mM.

FIG. 5C is a representative 96-well plate depicting the hemolysis according to the reaction described in FIG. 5B.

FIG. 6 is a plot chart illustrating that zinc gluconate inhibits hemolysis induced by Chironex fleckeri venom in whole blood.

FIG. 7 is a bar chart illustrating that zinc gluconate reduces the production of the potent pro-inflammatory cytokines PDGF-AA, EGF, G-CSF, GRO, IFNα₂, and TNFα in whole blood, which occur in response to Chironex fleckeri venom.

FIG. 8 is a bar chart showing that zinc gluconate reduces whole blood catecholamine and histamine plasma release, which increases in response to Chironex fleckeri venom.

FIG. 9 is a representative readout of a simultaneous echocardiogram/electrocardiogram recording for a mouse that was injected with a Chironex fleckeri venom.

FIG. 10 is a representative readout of a simultaneous echocardiogram/electrocardiogram recording for a mouse that was injected with a Chironex fleckeri venom and treated with zinc gluconate.

FIG. 11 is Kaplan-Meier plot illustrating the survival rates as well as duration of survival post-envenomation for all mice, untreated and treated with zinc gluconate, in a mouse study.

### DETAILED DESCRIPTION OF THE INVENTION

Pore-forming toxins, or porins, represent an ancient and conserved toxic exudate of most cnidarian venoms. In addition to characterizing the venom of cnidarian family members, porins are also a tool of infection used by pathogenic bacteria, including staphylococci, streptococci, anthrax, Clostridium, and E. coli. Potent membrane disruptive porins allow the evasion of host phagocytosis in bacterial infection and rapid prey cytolysis in invertebrate envenomation. They constitute a fundamental mechanism for infection and prey capture.

Porin structure and pore formation have been characterized by negative stain electron microscopy and other biochemical techniques that demonstrate the transition of plasma soluble, monomeric forms of these toxins to monomeric or polymerize to form oligomeric transmembrane pores remarkably comparable to the oligomeric form of human complement C9 (Borsos et. al. 1964. Lesions in erythrocyte membranes caused by immune haemolysis. Nature 202:251-252; Bhakdi, S. and Tranum-Jensen, J. 1985. Membrane damage by channel-forming proteins: staphylococcal alpha-toxin, streptolysin-O and the C5b-9 complement complex. Biochem. Soc. Symp. 50:221-233, each of the foregoing which is incorporated herein by reference in its entirety) as well as the human perforin, the cytolytic protein of cytotoxic T-cells (Young, et al. 1986. The ninth component of complement and the pore forming protein (perforin 1) from cytotoxic T cells: structural, immunological, and functional similarities. Science 233:184-190, which is incorporated herein by reference in its entirety).

Porin insertion compromises the permeability barrier of the cell membrane (Bashford, et al. 1985. Sequential onset of permeability changes in mouse ascites cells induced by Sendai virus. Biochim. Biophys. Acta 814:247-255; Bashford, et al. 1986. Membrane damage by hemolytic viruses, toxins, complement, and other cytotoxic agents. A common mechanism blocked by divalent cations. J. Biol. Chem. 261: 9300-9308) to result in membrane depolarization if pores allow monovalent ions passage. Specifically, efflux of K⁺, influx of Na⁺, influx of Ca²⁺ and efflux of Cl⁻ together result in depolarization of the cells as the internal and external ionic solutions rapidly equilibrate. Pores of sufficient size and open time duration will also allow diffusion loss of larger molecules such as intermediates of metabolism (e.g., nucleotides and sugar phosphates) to result in.a loss of cellular function, known as pyroptosis. Finally, large proteins such as hemoglobin, in the case of red blood cells (RBC), and lysosomal enzymes leak to result in hemolysis and tissue necrosis.

In conducting research on the systematic biochemical characterization of the venoms of the cubozoan members of the cnidarian families, it has been discovered that zinc is a rapid and effective inhibitor of several specific porin-related pathogenic outcomes, including, but not limited to, hyperkalemia, hemolysis, cytokine and histamine release, and catecholamine surge. Furthermore, zinc increases survival time following ethal doses of Chironex fleckeri (the Australian box jellyfish) venom in mice.

Chironex venom, as do all cubozoan venoms studied to date, contains an extremely potent pore forming toxin (PFT, also known as porin, cytolysin or hemolysin). These hemolysins have not been considered to be lethal, as clinical presentations post mortem did not show lethal levels of hemolysis. However, it has been discovered that a catastrophic hyperkalemic state precedes clinically measurable hemolysis, and furthermore, that this catastrophic hyperkalemic state is specifically caused by the cubozoan venom PFTs. Zinc-containing compounds, such as gluconate, have been discovered to inhibit hemolysis and the hyperkalemia that precedes hemolysis. Pore-forming toxins exhibit general classes of conserved structural homology for which some calcium appears to be involved in polymerization to form transmembrane pores. Thus, some divalent cations, such as, for example, Zn²⁺ and Mg²⁺, are able to competitively bind to calcium binding sites and inhibit self assembly of porin proteins to form functional polymeric pores.

Accordingly, in embodiments of the invention, methods for use of a composition that includes a zinc-containing compound in treating a disease or condition resulting from porin exposure are provided.

In embodiments of the invention, methods for treatment of a condition caused by cnidarian toxin poisoning are provided, in which the methods include administration of a composition that includes a zinc-containing compound in a therapeutically effective dose to a subject suffering from the condition. In some embodiments, the composition is administered intravenously.

In some embodiments, the zinc-containing compound is zinc gluconate.

Conditions and Diseases Associated with Exposure to Pore-Forming Toxins

In embodiments of the invention, pore-forming-toxin-related illnesses and conditions can include, but are not limited to, hyperkalemia, hypovolemia, hypocalcemia, toxic calcium influx, hemolysis, cytokine and histamine release, Irukandji syndrome, catecholamine surge, bacterial sepsis, cardiovascular collapse, pulseless electrical activity (PEA) and envenomation by cnidarians with cardiovascular collapse, respiratory distress, inflammation and/or Irukandji syndrome. For example, severe envenomations by the Carybdeidae family (a cubozoan member of the cnidarian phylum) can lead to Irukandji syndrome, which is associated with “catecholamine surge” and “cytokine storm” type symptoms that include pain, sweating, acute anxiety, and life threatening cardiovascular effects.

Additional conditions include diseases, illnesses or syndromes associated with porin-mediated cell and tissue damage, including, but not limited to, those resulting from a bacterial infection, a viral infection, a reaction to insect stings and arachnid bites, and reactions to stings from organisms within the cnidarian phylum.

Exemplary bacteria that produce PFTs of prominent health importance include, but are not limited to, Staphylococcus, Clostridium, Streptococcus, Bacillis, Aeromonas, Escherichia, and Neisseria.

Exemplary viruses that produce PFTs of prominent health importance include, but are not limited to, viruses from the Reoviridae, Paramyxoviridae and Orthomyxoviridae families.

Members of the cnidarian phylum that produce PFTs of prominent health importance include, but are not limited to, cubozoans (or box jellyfish), Physalia sp., stinging nettles, anemones, coral, fire coral and stinging hydroid.

Pore-forming toxin related illnesses and conditions can be caused by the exemplary agents listed in Table 1.

TABLE 1
Agents that form divalent cation-sensitive pores
(from: Bashford, C. L. Membrane Pores - From Biology to
Track-Etched Membranes. Biosci. Rep. 15 (1995) 553-565.)

| Agent | Examples |
|---|---|
| Viruses | Sendai, Newcastle Disease, Influenza |
| Bacterial toxins | S. aureus α- and δ-toxin, Streptolysin O, C. |
| | perfringens q-toxin, S. pneumoniae pneumolysin, |
| | E. coli haemolysin, A. hydropohila |
| | haemolysin, C. lacteus cytolysin, B. thuringiensis |
| | δ-endotoxin |
| Animal toxins | Melittin (honey bee), Cytolysin (anthozoans, |
| | cubozoans), Latrotoxin (spider venom) |
| Immune proteins | Activated complement, Cytolysin (perforin) |
| Synthetic | Polycations, Triton X-100 |
| compounds | |

Pore-forming toxin related illnesses and conditions can also be caused by the family of pore-forming mushroom toxins. Exemplary toxins in this family include, but are not limited to, phallolysin, flammutoxin, ostreolysin, and the cytolytic proteins identified in Berheinmer (Bernheimer, A. W., and B. Rudy. 1986. Biochim Biophys Acta 864:123-141, which is incorporated herein by reference in its entirety).

Other medically relevant porin-mediated conditions or sources of porin exposure will be apparent to a person of ordinary skill in the art.

### Zinc-Containing Compounds

In embodiments of the invention, the zinc-containing compound can include any non-toxic counter-ion to zinc. For example, the counter-ion can be any sugar-based counter-ion, including, but not limited to, acetate, malate or formulations based on D-lactulose, glucose, lactose, galactose, sucrose, pentose, and fructose. In some embodiments, the counter-ion can be any anion, including, but not limited to, chloride, sulfate, phosphate, acetate, propionate, butyrate, oxalate, malonate, succinate, or a complex polyanion. In some embodiments, the zinc-containing compound can be zinc gluconate.

In some embodiments, the counter-ion can be any ion selected for its property in meeting a desire to 1) avoid placing an additional ionic load in the plasma and/or 2) avoid burdening the kidney clearance load of a subject afflicted by a porin-mediated disease or condition. Applicable counter-ions that meet these criteria. will be apparent to a person of ordinary skill in the art

### Dosage

The dosage administered will vary depending upon known factors, such as the pharmacodynamic characteristics of the particular agent; the age, health and weight of the recipient; the nature and extent of the symptoms; concurrent treatment; the frequency of treatment; and the effect desired. In addition, an effective amount of a composition that includes a zinc-containing compound will depend, at least, on the particular method of use, the subject being treated, the severity of the affliction, and the manner of administration of the composition. A “therapeutically effective amount” of a composition is a quantity of a specified compound sufficient to achieve a desired effect in a subject (host) being treated. For example, this can be the amount of a zinc-containing compound necessary to prevent, inhibit, reduce or relieve a condition caused by a pore-forming toxin as disclosed herein.

Therapeutically effective doses of a disclosed zinc-containing compound or pharmaceutical composition containing the same can be determined by one of skill in the art. The amount of the compound or the pharmaceutical composition containing the same that is effective in the treatment or prevention of a condition associated with a pore-forming toxin can be determined by standard clinical techniques well known to those of skill in the art. In addition, in vitro or in vivo assays can optionally be employed to help identify optimal dosage ranges. One of ordinary skill in the art will readily be able determine the precise dose to be employed. Suitable daily effective dosage amounts, however, typically range from about 0.001 mg/kg of body weight to about 250 mg/kg of body weight, from about 0.01 mg/kg of body weight to about 100 mg/kg of body weight, from about 0.1 mg/kg of body weight to about 50 mg/kg of body weight, or from about 1 mg/kg of body weight to about 25 mg/kg of body weight. The effective dosage amounts described herein refer to total amount of zinc-containing compound administered.

In some embodiments, a therapeutically effective dose of a disclosed zinc-containing compound, or pharmaceutical composition containing the same, is a circulating dose of between about 1mM and about 10 mM, between about 2 mM and about 8 mM, or between about 4 mM and 6 mM. In some embodiments, a therapeutically effective dose is a circulating dose of about 5 mM.

Pharmaceutical formulations

In embodiments of the invention, a therapeutic treatment is provided, the treatment comprising the use of a zinc-containing compound as disclosed herein, a pharmaceutical composition or therapeutic agent containing the same, or a pharmaceutically acceptable salt or solvate thereof, and at least one pharmaceutical carrier or. diluent. The compound or composition can be used in the prophylaxis and/or treatment of the foregoing diseases or conditions and in therapies as disclosed herein. In some embodiments, the carrier is a pharmaceutically acceptable carrier and is compatible with, i.e. does not have a deleterious effect upon, the other ingredients in the composition. The carrier can be a solid or liquid and can be formulated as a unit dose formulation, for example, as a tablet that can contain from 0.05 to 95% by weight of the active ingredient.

In some embodiments, the zinc-containing compound is present in the pharmaceutical composition in an amount ranging from about 0.5 percent to about 90 percent by weight of the pharmaceutical composition. In some embodiments, the zinc-containing compound is present in the pharmaceutical composition in an amount ranging from about 1 percent to about 85 percent by weight of the pharmaceutical composition. In some embodiments, the zinc-containing compound is present in the pharmaceutical composition in an amount ranging from about 5 percent to about 80 percent by weight of the pharmaceutical composition. In some embodiments, the zinc-containing compound is present in the pharmaceutical composition in an amount ranging from about 10 percent to about 75 percent by weight of the pharmaceutical composition. In some embodiments, the zinc-containing compound is present in the pharmaceutical composition in an amount ranging from about 15 percent to about 50 percent by weight of the pharmaceutical composition. In some embodiments, the zinc-containing compound is present in the pharmaceutical composition in an amount ranging from about 25 percent to about 35 percent by weight of the pharmaceutical composition.

In some embodiments, the zinc-containing compound is present in the pharmaceutical composition in an amount ranging from about 2 percent to about 25 percent by weight of the pharmaceutical composition. In some embodiments, the zinc-containing compound is present in the pharmaceutical composition in an amount ranging from about 2 percent to about 20 percent by weight of the pharmaceutical composition. In some embodiments, the zinc-containing compound is present in the pharmaceutical composition in an amount ranging from about 2 percent to about 10 percent by weight of the pharmaceutical composition. In some embodiments, the zinc-containing compound is present in the pharmaceutical composition in an amount ranging from about 5 percent to about 15 percent by weight of the pharmaceutical composition. In some embodiments, the zinc-containing compound is present in the pharmaceutical composition in an amount ranging from about 5 percent to about 10 percent by weight of the pharmaceutical composition.

In some embodiments, the pharmaceutical composition is a solution.

In some embodiments, the pharmaceutical composition is injectable.

In some embodiments, the pharmaceutical composition can be administered parenterally.

In some embodiments, the pharmaceutical composition can be administered topically, such as, for example, by a solution, a spray, a lotion, or an ointment.

In some embodiments, the pharmaceutical composition can be administered subcutaneously.

In some embodiments, the pharmaceutical composition can be administered intravenously.

In some embodiments, the pharmaceutical composition can be administered orally.

In some embodiments, the pharmaceutical composition can be administered intramuscularly.

In some embodiments, the pharmaceutical composition can be administered intraperitoneally.

In some embodiments, the pharmaceutical composition can be administered transdermally, such as, for example, by a transdermal patch.

In some embodiments, the therapeutic zinc composition formulation can contain at least one additional agent, including, but not limited to, a carrier, an adjuvant, an emulsifying agent, a suspending agent, a sweetener, a flavoring, a perfume, a binding agent, or the like.

As used herein, “pharmaceutically acceptable carrier” and “carrier” generally refer to a non-toxic, inert solid or non-inert, semi-solid or liquid filler, diluent, encapsulating material or formulation auxiliary of any type. Some non-limiting examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil; kukui nut oil, camphor oil; and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, menthol and perfuming agents, preservatives and antioxidants can also be present in the composition, according to the judgment of the formulator.

The pharmaceutically acceptable carriers described herein, for example, vehicles, adjuvants, excipients, or diluents, are well-known to those who are skilled in the art. Typically, the pharmaceutically acceptable carrier is chemically inert to the therapeutic agents and has no detrimental side effects or toxicity under the conditions of use. The pharmaceutically acceptable carriers can include polymers and polymer matrices, nanoparticles, microbubbles, and the like.

The therapeutic treatment can further comprise inert diluents such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

### Routes and Forms of Administration

The zinc-containing compound, or a therapeutic composition containing the same, can be delivered by a variety of routes, including, but not limited to, intravenous, intramuscular, topical, and oral, that can be optimized to the clinical scenario. Additional routes of administration routes include sublingual, buccal, parenteral (including, for example, subcutaneous, intramuscular, intra-arterial, intraperitoneal, intracisternal, intravesical, intrathecal, or intravenous), transdermal and rectal points of entry.

In some embodiments, the zinc-containing compound is administered transdermally. Typically, the compound is applied to the drug electrode of an iontophoresis unit, and the drug electrode and ground electrode are applied to the skin of a subject in need of treatment. Voltage is then applied to deliver the compound transdermally to the subject. Typical compound concentrations applied to the drug electrode range from about 0.1 mM to about 250 mM, from about 0.5 mM to about about 200 mM, from about 1 mM to about 100 mM, from about 2.5 mM to about 50 mM, or from about 5 mM to about 25 mM. Typical voltages applied to the skin of the subject range from about 0.1 mAmp/min to about 80 mAmp/min. An appropriate voltage amount is one that alleviates symptoms associated with exposure to a pore-forming toxin and the improves medical outcome for the subject while maintaining the comfort level of the subject being treated.

The methods of treatment of the present invention include methods that are administered to a subject in need thereof. As used herein, “subject,” may refer to any living creature, typically an animal, preferably a mammal, and more preferably a human.

Formulations suitable for oral administration can be provided as discrete units, such as tablets, capsules, cachets, syrups, elixirs, chewing gum, “lollipop” formulations, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Formulations suitable for transmucosal methods, such as by sublingual or buccal administration include lozenges patches, tablets, and the like comprising the active compound and, typically a flavored base, such as sugar and acacia or tragacanth and pastilles comprising the active compound in an inert base, such as gelatin and glycerine or sucrose acacia.

Formulations suitable for parenteral administration typically comprise sterile aqueous solutions containing a predetermined concentration of the zinc composition and possibly another therapeutic agent; the solution is preferably isotonic with the blood of the intended recipient. Additional formulations suitable for parenteral administration include formulations containing physiologically suitable co-solvents and/or complexing agents such as surfactants and cyclodextrins. Oil-in-water emulsions may also be suitable for formulations for parenteral administration of the gas-enriched fluid. Although such solutions are preferably administered intravenously, they may also be administered by subcutaneous or intramuscular injection.

Formulations suitable for transdermal administration can be prepared for delivery by transdermal patches with or without electrophoretic current to augment diffusion or deliver agent. Transdérmal administration can be also by use of “nanoneedles”. (see Escobar-Chávez J J, Bonilla-Martinez D, Villegas-González M A, Revilla-Vázquez A L. J Clin. Pharm (2009), which is incorporated by reference in its entirety).

Formulations of the invention can be prepared by any suitable method, typically by uniformly and intimately admixing the zinc-containing compound optionally with liquids or finely divided solid carriers or both, in the required proportions and then, if necessary, shaping the resulting mixture into the desired shape.

For example, a tablet can be prepared by compressing an intimate mixture comprising a powder or granules of the zinc-containing compound and one or more optional ingredients, such as a binder, lubricant, inert diluent, or surface active dispersing agent, or by molding an intimate mixture of powdered zinc-containing compound of the present invention.

In addition to the ingredients specifically mentioned above, the formulations of the present invention can include other agents known to those skilled in the art, having regard for the type of formulation in issue. For example, formulations suitable for oral administration can include flavoring agents and formulations suitable for intranasal administration may include perfumes.

In some embodiments, the zinc-containing compound is used as a prophylactic treatment prior to a subject coming in contact with an agent that causes the reactions, symptoms or conditions disclosed herein. In some embodiments, the zinc-containing compound is used as a prophylactic treatment prior to a subject encountering a cnidarian.

Suitable pharmaceutical carriers are described in Remington's Pharmaceutical Sciences, Mack Publishing Company, a standard reference text in this field.

Combination therapy

In embodiments of the invention, the methods disclosed herein further comprise a combination therapy, wherein at least one additional therapeutic agent is administered to the patient. In some embodiments, the at least one additional therapeutic agent is selected from the group consisting of antibiotics (such as penicillin, tetracycline, and Tobramycin) to control infection, D-lactulose, specific phospholipase inhibitors useful in certain types of envenomations, steroids, and pain relievers/anti-inflammatory agents (such as ibuprofen).

The therapeutic methods of the invention can be administered by any conventional method available for use in conjunction with pharmaceutical drugs, either as individual therapeutic agents or in a combination of therapeutic agents.

It will be appreciated that the methods of the combining zinc gluconate with an additional treatment can be administered: (1) simultaneously by combination of the compounds in a co-formulation or (2) by alternation, i.e. delivering the compounds serially, sequentially, in parallel or simultaneously in separate pharmaceutical formulations. In alternation therapy, the timing of administration of the second, and optionally a third active ingredient, is such that there is no loss of benefit of any synergistic therapeutic effect of the combination of the active ingredients. In some embodiments, by either method of administration (1) or (2), the combination is preferably administered to achieve the most efficacious results. In some embodiments, by either method of administration (1) or (2), the combination is administered to achieve peak plasma concentrations of each of the active ingredients.

In some embodiments, D-lactulose can substantially inhibit hemolytic toxins. In particular, a dramatic absence of hemolysis was observed in the presence of 10 mM D-lactulose. (Chung, J. J., Ratnapala, L. A., Cooke, I. M., Yanagihara, A. A., Toxicon 39 (2001) 981-990, hereby incorporated by reference in its entirety). In some embodiments, a zinc-containing composition is used in combination with D-lactulose, wherein the presence of D-lactulose results in an increased inhibition of the pore-forming toxin and thus substantially reduces morbidity and mortality.

In some embodiments, the amount of D-lactulose administered to a subject afflicted by a porin-mediated condition is between about 1 mM and about 50 mM, between about 2 mM and about 25 mM, between about 5 mM and about 15 mM, or between about 7.5 mM and about 12.5 mM. In some embodiments, the amount of D-lactulose administered to a subject afflicted by a porin-mediated condition is about 10 mM.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

A Zinc-Containing Compound Inhibits the Effects of Cnidarian Venom in Whole Blood and Isolated Red Blood Cells

Concentration gradient driven monovalent ion flux, or more specifically, potassium (K⁺) efflux into plasma accompanied by both sodium (Na⁺) influx and chloride ion (Cl⁻) efflux, as well as divalent cation toxic influx (Ca²⁺), can occur in cubozoan envenomation with sufficiently rapid kinetics, and this can lead to cardiotoxic calcium influx with lethal plasma hyperkalemia at rates beyond the kidney clearance rate in an affected subject. Ex vivo assays of whole human blood demonstrated that profound hyperkalemia results from Chrionex PFT exposure with lethal levels of free plasma potassium (>10 mM). A zinc ionic compound containing a carbohydrate counterion, was used for the studies of cubozoan PFT inhibition, and zinc inhibitory effects were tested on cnidaria-porin associated pathogenic processes.

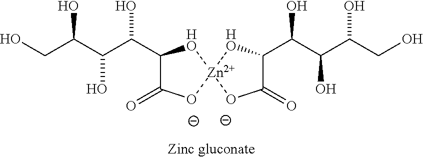

Whole blood or human washed red blood cells (RBC) were subjected to either Carybdea alata (CA) or Chironex fleckeri (CF) venom or purified porin from Carybdea alata (CA) or Chironex fleckeri (CF). These cells were treated simultaneously with a 1/20 total volume dose of 100 mM zinc gluconate to achieve a final concentration of 5 mM, and the time of potassium efflux was continuously determined by ion specific electrode.

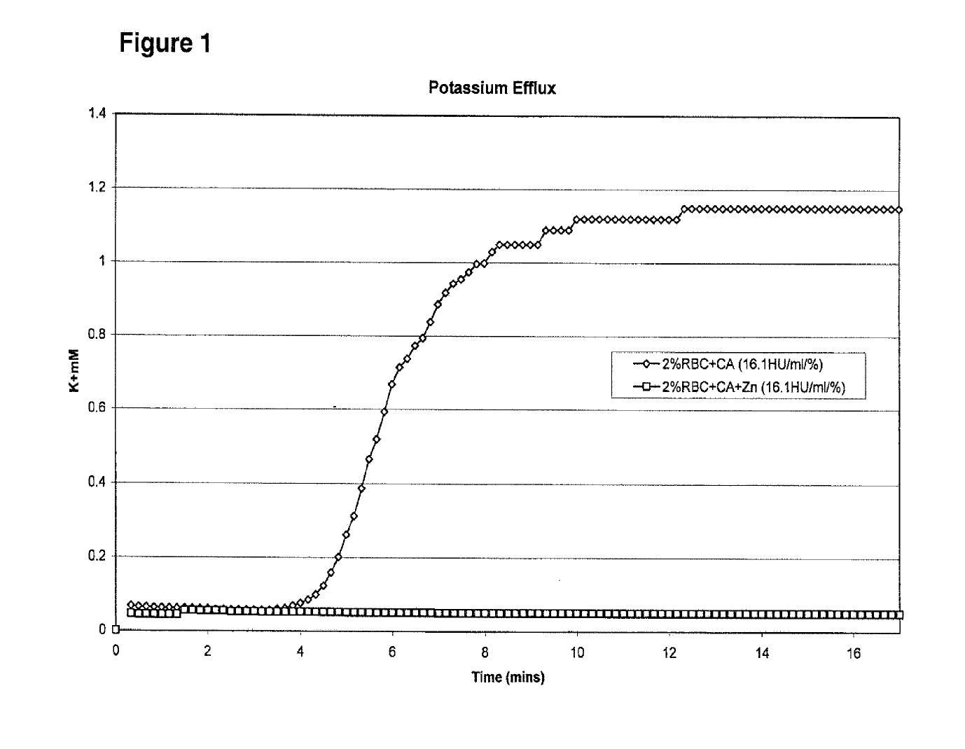

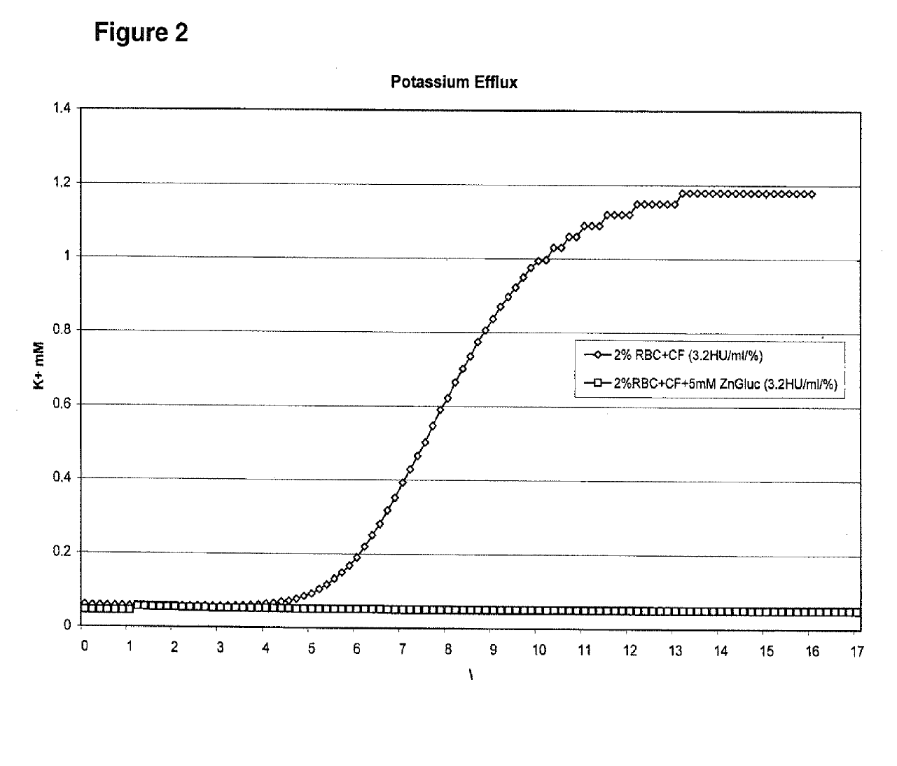

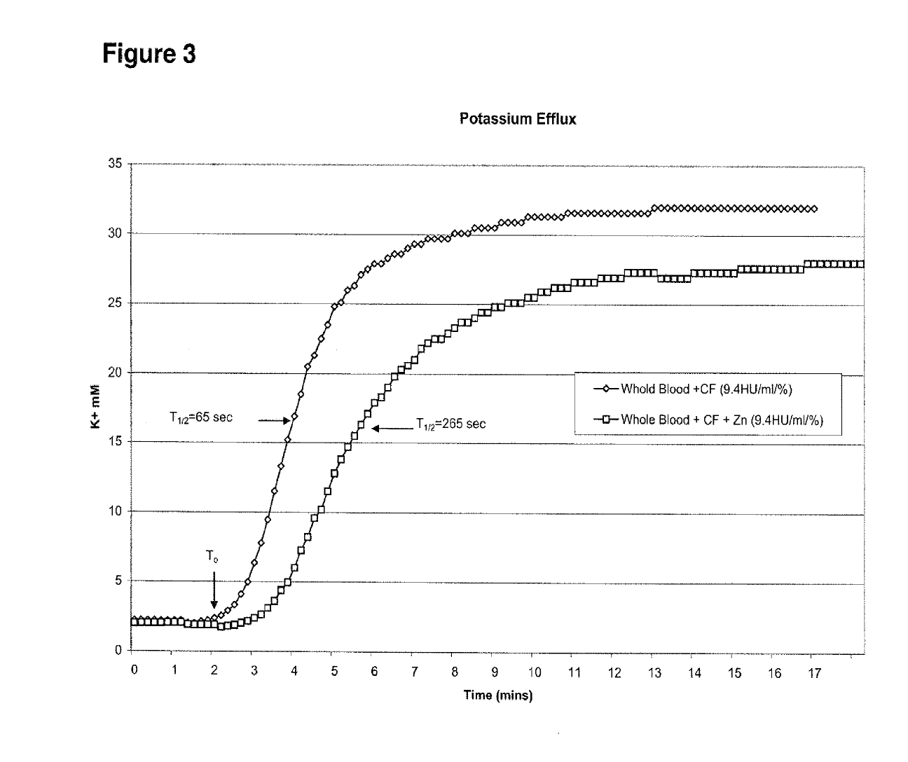

FIGS. 1 and 2 illustrate the results of these experiments. Venom amount is provided in U/ml/% where one unit is equivalent to the amount of venom that lyses a 1% RBC solution in one hour at 37° C. As shown in FIG. 1, zinc gluconate fully inhibited potassium efflux induced by Carybdea alata venom in 2% RBC. As shown in FIG. 2, zinc gluconate fully inhibited potassium efflux induced by Chironex fleckeri venom in 2% RBC. As shown in FIG. 3, zinc gluconate inhibited potassium efflux induced by Chironex fleckeri venom using whole blood. The results thus indicate that a zinc-containing compound is effective in counteracting the ion flux caused by exposure to a pore-forming toxin.

### EXAMPLE 2

A Zinc-Containing Compound Reduces Porin-Mediated-Hemolysis in Whole Blood and Isolated Red Blood Cells

Whole blood or human washed red blood cells (RBC) were subjected to either Carybdea alata (CA) venom or purified hemolysin. These cells were treated simultaneously with a 1/20 total volume dose of 100 mM zinc gluconate to achieve a total final concentration of 5 mM. Aliquots were removed at each time point and pulse microfuge spun for plasma separation. Plasma hemoglobin levels at each time point were then determined spectrophotometrically.

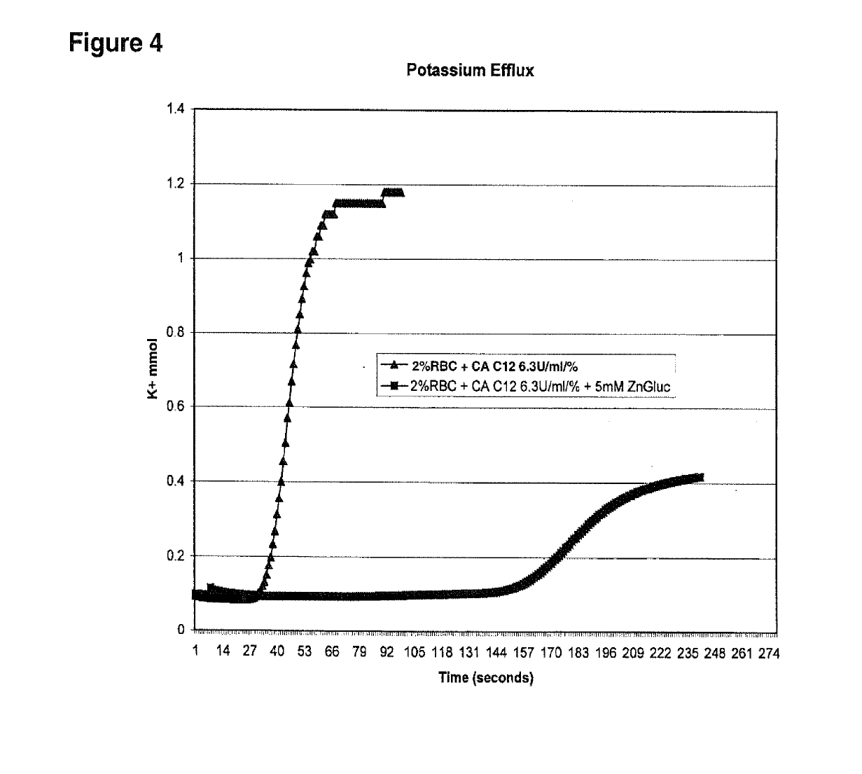

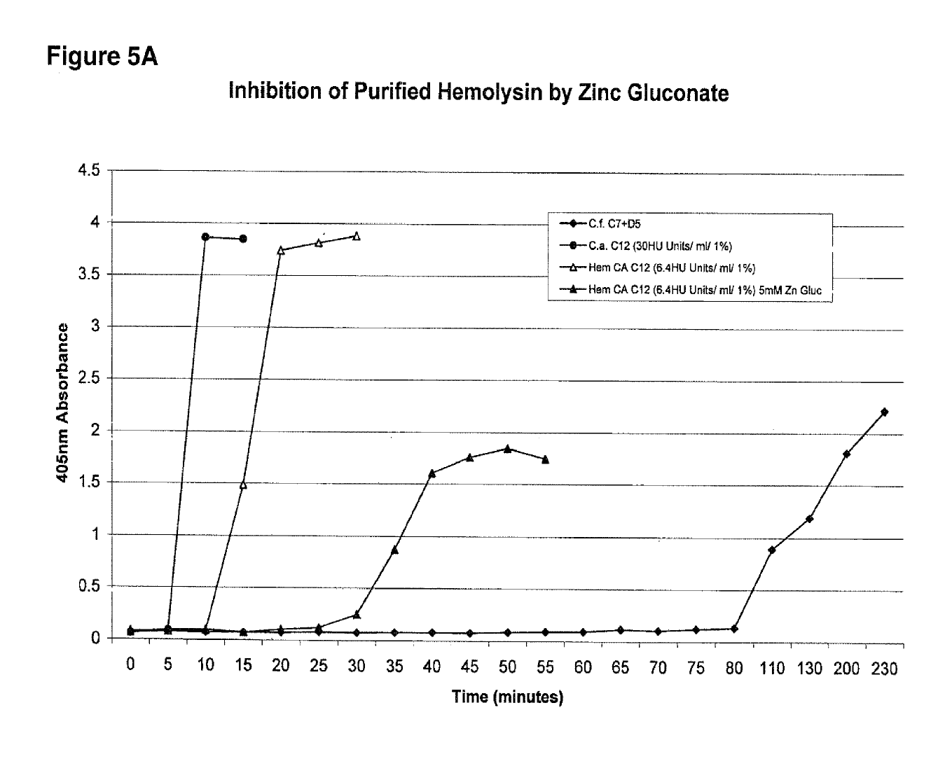

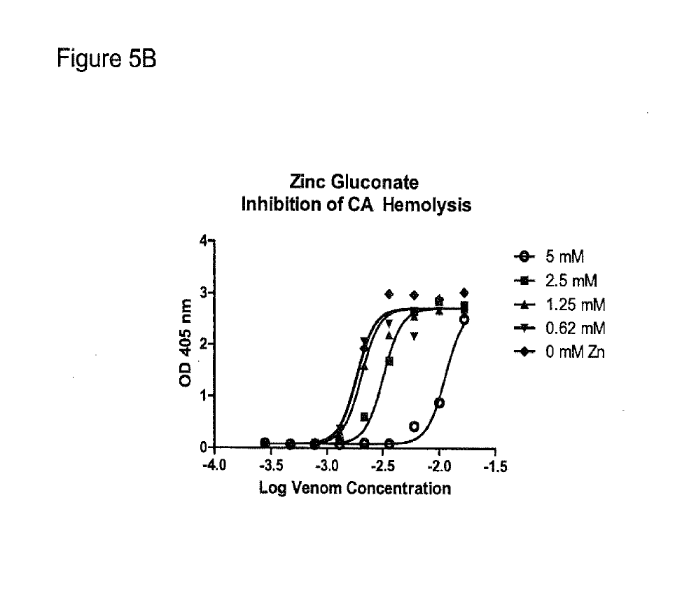

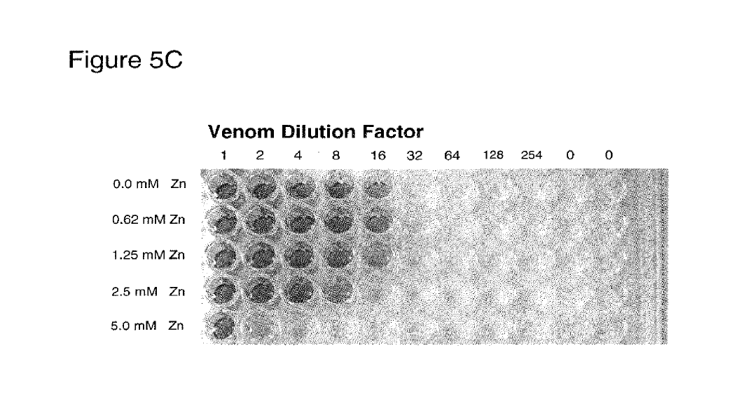

As shown in FIG. 4, zinc gluconate substantially inhibited hemolysis induced by Carybdea alata venom in 2% RBC. As shown in FIG. 5, zinc gluconate substantially inhibited hemolysis induced by Carybdea alata hemolysin in 2% RBC. In particular, zinc gluconate slowed the T½ of lysis from 10 minutes to 40 minutes and decreased the total hemolytic capacity of the venom dose (6.4 U/ml) by more than 10 fold. The results thus indicate that a zinc-containing compound is effective in inhibiting or delaying onset of hemolysis caused by exposure to a pore-forming toxin.

### EXAMPLE 3

A Zinc-Containing Compound Improves Porin-Mediated-Cytokine Response of Whole Blood

In this example, whole blood was subjected to Chironex fleckeri (CF) venom in the presence or absence of 5 mM zinc gluconate, and production of cytokines in the cells was determined.

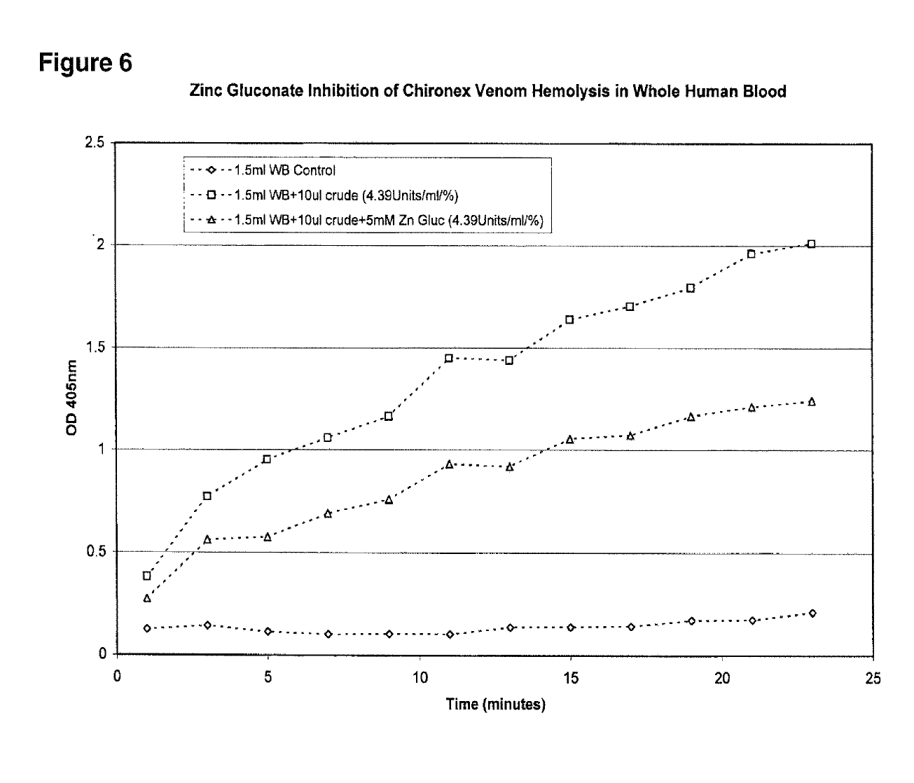

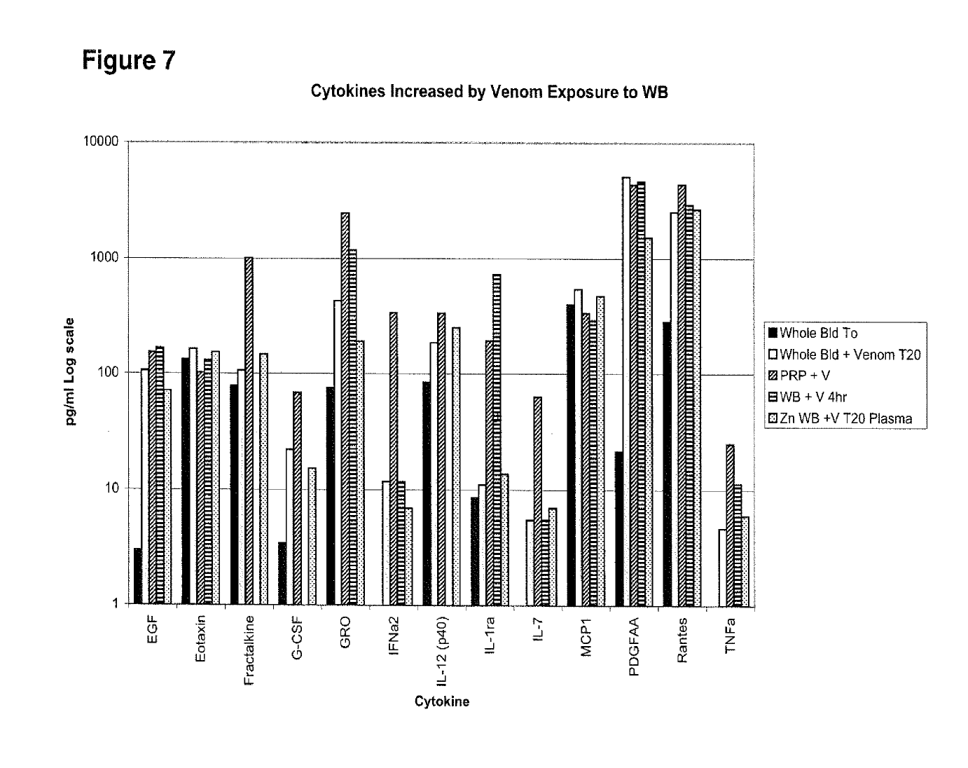

As shown in FIG. 6, the zinc-gluconate-treated cells exhibited substantially less severe hemolysis compared to untreated cells. As shown in FIG. 7, zinc gluconate substantially altered the production of cytokines induced by Chironex fleckeri (CF) venom in whole blood samples. In particular, the production of potent pro-inflammatory cytokines PDGF-AA, EGF, G-CSF, GRO, IFNα₂, and TNFα were reduced. The marked reduction in the release of these potent inflammatory chemo attractants upon the inclusion of zinc gluconate in CF venom exposed blood indicates the utility of a zinc compound in the treatment of cubozoan envenomation associated “cytokine storm” response of Irukandji syndrome.

### EXAMPLE 4

A Zinc-Containing Compound Reduces Porin-Mediated-Catecholamine and Histamine Response in Whole Blood

Whole blood was subjected to Chironex fleckeri (CF) venom. The toxin-exposed cells were then treated simultaneously with 5 mM zinc gluconate and the catecholamine and histamine response was determined.

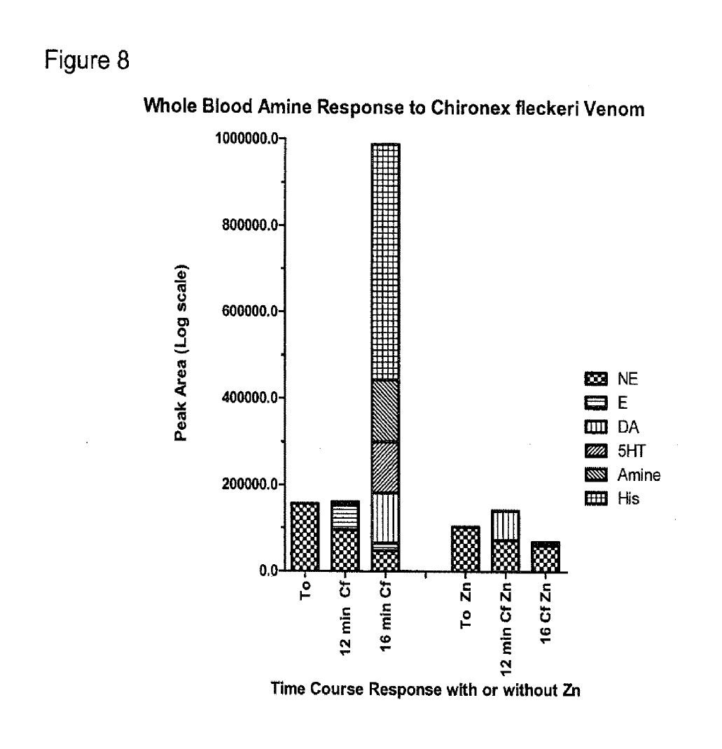

As shown in FIG. 8, zinc gluconate reduced the catecholamine and histamine response induced by Chironex fleckeri (CF) venom in whole blood samples. Thus, the results indicate that a zinc-containing compound can ameliorate the catecholamine and histamine response associated with exposure to a pore-forming toxin. These results are indicative of an improved clinical outcome when zinc gluconate is administered to a subject suffering from a condition caused by porin exposure.

### EXAMPLE 5

A Zinc-Containing Compound Increases Survival Time in Mice Exposed to A Pore-Forming Toxin

Murine subjects were treated with zinc gluconate intravenously while simultaneously being envenomated with Chironex fleckeri venom. In treated mice, zinc gluconate was administered as a single bolus two minutes prior to envenomation as well as one minute post-envenomation. Untreated mice did not receive any zinc gluconate. All mice were envenomated with an isolate of tentacle-free, highly purified mastigophore (penetrant cnidae) total Chironex venom. The mice were then observed to determine the effects of zinc gluconate administration.

Table 2 indicates the mouse intravenous tail vein response to Chironex fleckeri venom with and without zinc gluconate. As illustrated in Table 2, the use of zinc gluconate increased the survival time of the murine subjects up to 12 hours. In the absence of zinc gluconate treatment, the mice died within minutes of envenomation. The results indicate that administration of a zinc-containing compound can improve survival time in a subject exposed to a pore-forming toxin.

TABLE 2
Effect of zinc gluconate pre- and post-treatment on the survival time
in mice in IV tail vein injections of Chironex fleckeri venom

| Lethal Dose | x | x | X | |
|---|---|---|---|---|
| Animal # | 1 | 2 | 3 | |
| Animal mass (g) | 31 | 32 | 36 | |
| Pretreat 100 mM | | | T = −2 min | 150 ul |
| ZnGluc | | | | |
| Total Venom | ~80 ul | 100 ul | 100 ul | |
| Units/μL | | | | |
| (2633 U/100 ul) | | | | |
| Post 100 mM Zinc | | | T = 1 min | 150 ul |

| | | | |
|---|---|---|---|
| Death time | 6 min | 1 min | 12 hours |

### EXAMPLE 6

A Zinc-Containing Compound Improves Cardiovascular Abnormalities in Mice Exposed to A Pore-Forming Toxin

A mouse model was developed to study the mechanisms underlying life-threatening cardiovascular collapse resulting from Chironex fleckeri (Australian sea wasp or box jellyfish) envenomation in humans.

### Protocols

Cnidae Isolation

Carybdea alata. Freshly beached, post-spawning Alatina moseri were collected in the early-morning hours along specific leeward Oahu (Hawaii) beaches during synchronized spawning cycles, occurring 8-10 days after each full moon. Tentacles were excised beachside and placed immediately into chilled 1 M citrate at approximately 1:4 (v:v) in 50-mL tubes and agitated at 4° C. for up to 8 weeks to recover all tentacular cnidae through a process of hypertonic mesogleal tissue contraction and intact cnidae sloughing. Contents were sieved (using 0.5-mm plankton sieves) to recover undischarged cnidae from the cnidae-free tentacles.

Chironex fleckeri were collected in North Queensland Australia. Tentacles were excised beachside and frozen at −80° C. Aliquots of frozen tentacles were resuspended in 1 M citrate at approximately 1:20 (v:v) in 50-mL tubes and agitated at 4° C. for up to 2 weeks to recover all tentacular cnidae through a process of hypertonic mesogleal tissue contraction and intact cnidae sloughing. Contents were sieved (using 0.5-mm plankton sieves) to recover undischarged cnidae from the cnidae-free tentacles.

Purified Cnidae Venom Preparation. The sieved cnidae solution was centrifuged at 400 g for 20 minutes; pellets were resuspended in 1 M citrate at 1:20 (v:v) and centrifuged two more times at 250 g for 20 minutes. Cnidae were counted using a hemocytometer (KOVA, glasstic with grids, HYCOR 87144) at each step of the preparation. After the final spin, the pellet was diluted 1:0.5 (v:v) with near 0° C. deionized water and immediately placed in a pre-chilled (ice water bath) French Press 20 K pressure cell (SLM-AMINCO Cat# FA078 Serial #9003402). The cell was pressurized at 750 on setting HIGH (total approximately 12000 psi) for 10-15 minutes or with a flow of approximately 30 drops/min to disrupt the cnidae and thus recover total cnidae contents or “total venom.” This process was repeated rapidly for 2-4 passes to achieve >95% cnidae rupture. The total venom was aliquotted into 1.5-mL microfuge tubes and centrifuged at 12,000 g for 5 minutes. The supernatant was filtered (using Millipore 0.45 mm PVDF filter membrane), and aliquotted into 100-mL volumes, snap frozen in liquid nitrogen and then stored at −80° C.

Protein Determination. Sample protein concentrations were determined by the method of Bradford (1976) using the Bio-Rad Protein Assay Kit (Bio-Rad) by comparison with bovine serum albumin (BSA) protein concentration standards.

Hemolytic Activity Assay. A hemolytic activity assay, modified from the protocol of Hessinger and Lenhoff (Hessigner, D. A. and H. M. Lenhoff. 1973. Arch Biochem Biophys 159:629-638, which is incorporated herein by reference in its entirety), was carried out in a 96-well arrow-bottom microtiter plate using 2% blood drawn from healthy human donors washed three times with phosphate buffered saline (PBS) (136.9 mM NaCl, 2.68 mM KCl, 10.14 mM Na₂HPO₄, and 1.76 mM KH₂PO₄, pH 7.4). Washing was done by low-speed centrifugation (500×g, 10 min) of the blood at 4° C. A 1:1 serial dilution of the total venom was carried out over two rows in the 96-well plate using saline as the diluent. Subsequently, 170 mL of the 2% blood solution was added to each of the 20 mL sample wells, and the plate was incubated at 37° C. for 60 minutes. Plates were then centrifuged (1500 g for 10 min) and supernatants from each of the wells were transferred to a 96-well flat bottom microtiter plate for absorbance determination measured using a Biorad Ultramark microplate (Bio-Rad, Hercules, CA) reader at 405 nm. Reference samples were employed using hypotonic lysis with water as a 100% lysis reference and the 2% blood alone as the 0% reference. Where quantitative measurements of hemolysis are shown, samples were assayed in the same experiment with the same diluted blood and the same batch of isolated venom. An HU₅₀ unit is defined as that amount of protein required to lyse 50% of the red blood cells in a 1-mL volume of a 1% blood solution at 37° C. in one hour. Though the exact amount varied with monthly jellyfish captures, an HU₅₀ unit typically represented about 20 ng total venom protein.

Echocardiography/Electrocardiography Testing. C57B⅙ mice, weighing approximately 17-28 g, were anaesthetized using Isoflurane (3%) with oxygen for 2 minutes, placed dorsally onto a thermostat-regulated heated platform with paws secured by surgical tape at built-in electrocardiography (ECG) electrodes. The body temperature was regulated at 37° C. and the flow of Isoflurane (1%) and oxygen were maintained via a nose cone and monitored throughout the procedure to ensure a continuous sedated state. The hair on the upper left torso was removed and a 30-MHz transducer was placed on the left hemithorax for transthoracic echocardiography (ECHO) using a Vevo 770 (Visualsonics, Toronto, Canada). Care was taken to avoid excessive pressure when the ECHO M-mode cursor was positioned over the aortic root, and the view of the mid-left ventricular (LV) was then located at the chordal level by rotating the transducer clockwise 30-45° . Images from this view were used to measure LV fractional shortening and aortic contractions. Mouse tail-vein catheters (SAI Infusion Technologies, MTV-01) were inserted into the tail vein and up to five pre-injection readings were taken to ensure a steady ECG and LV-ejection fraction base. Injection volumes of zinc and/or total venom were calculated by animal weight and administered at a flow of 200 μL/min, followed by saline (150 mM NaCl) to wash and maintain catheter volume control. High-resolution M-mode ECHO and ECG data were simultaneously recorded at 100 mm/5 second sweeps and stored in digital format. During the first 90 minutes of the procedure, all clinical signs and markedly altered behavior were closely monitored and recorded. Within 60 seconds of time of death as determined by loss of EKG activity and respiration or after CO₂ mediated euthanasia, cardiac blood was carefully drawn by 22 ½ gauge into an additive free sterile syringe and transferred to an microfuge tube. Whole blood samples were immediately centrifuged (6,000 g, 2 min, room temperature) to separate plasma. Plasma was stored at −80° C. until further assayed to determine hemoglobin and electrolyte levels.

Hemoglobin Quantitation. Plasma hemoglobin concentrations were determined using a plate reader method, as described above, by converting absorbance at 405 nm to concentration using Beer's law with a molar extinction coefficient ε of 276069 and molar mass (64,500 g/mol).

Plasma Potassium Quantitation. Plasma potassium concentrations were determined in triplicate serial dilutions of plasma using a double-junction ion-specific electrode (ELIT 8031 Potassium electrode, with Double Junction Reference Electrode 003N with an Nico 2000 LTD Middlesex, UK) and 4-channel Ion Analyser Software (Version 7.1.44sa, 2006) utilizing reference standard curves from 10 (0.26 mM) to 1000 ppm (26 mM) using authenticated potassium chloride (KCl) standards.

Data Analysis. LV fraction shortening was characterized by a point estimate of central tendency and corresponding 95% confidence intervals. Multivariate log-normal plots were used to assess underlying normality. When appropriate, a normalizing transformation was applied to the data. Hypothesis tests of the mean difference between the experimental and the control group were performed via the method of least-square means. Statistical tests were adjusted for multiplicity via the sequential Hochberg-Bonferroni method. P-values <0.05 were considered statistically significant. Based upon preliminary data, the sample of n=60 animals (5 per group) had 80% power to detect a mean difference of 10.11 at a significance level=0.05 (multiplicity corrected), given a standard deviation of the mean difference of 5.0 or less. All statistical analyses were performed using SAS software package (Cary, N.C.). Survival data were analyzed using GraphPad Prism software version 5.00 for Windows (GraphPad Software, San Diego California USA). Significant differences (p<0.05) between groups were determined using the χ2—test (Fisher's exact test was used instead of χ2 when only two groups were considered) and Kruskal-Wallis statistic (Mann-Whitney test was used instead of Kruskal-Wallis when only two groups were considered). Survival curves were analyzed according to the Kaplan-Meier method, and for differences between curves the p value was calculated by the log-rank test. A p value of less than 0.05 was accepted as statistically significant.

### Results

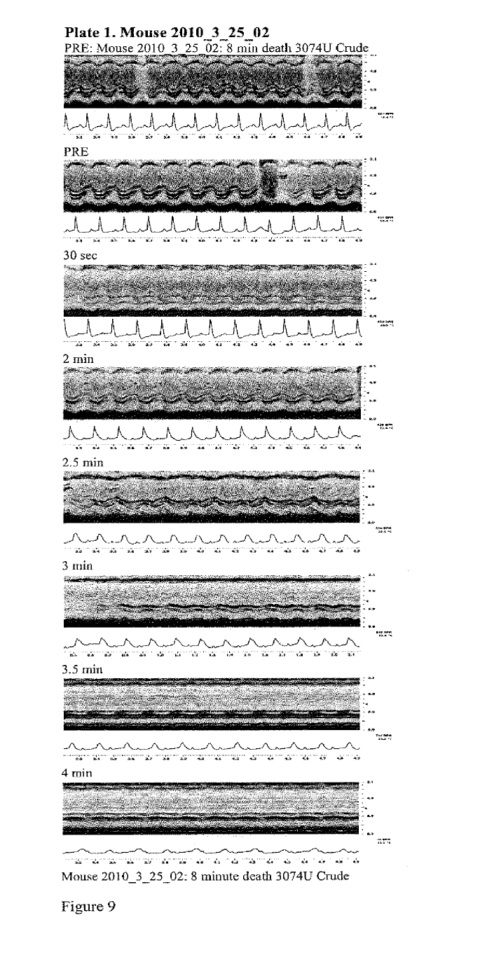

Continuous pre and post-injection recordings were performed for 30 minutes by ECHO of the left ventricle and paw-recorded EKG. Over the course of the study, over 200 mice were tested over a broad range of doses. FIG. 9 illustrates representative data from Chironex venom injected mice arranged according to dose and survival time. It was observed in the envenomated mice that, at higher doses (3000 U and over), total venom injection resulted in acute ventricular demise together with conductive system anomalies. Such responses are illustrated in FIG. 9 (Mouse 2010_{—}3_{—}25_{—}02) injected with a total of 3000 U or an approximate dose equivalent of a human lethal sting (3M contact). QRS widening at 2 minutes is followed by lack of effective left ventricular contraction. This pulseless electrical activity (PEA) occurred concomitant to EKG abnormalities. Death occurred at 8 minutes.

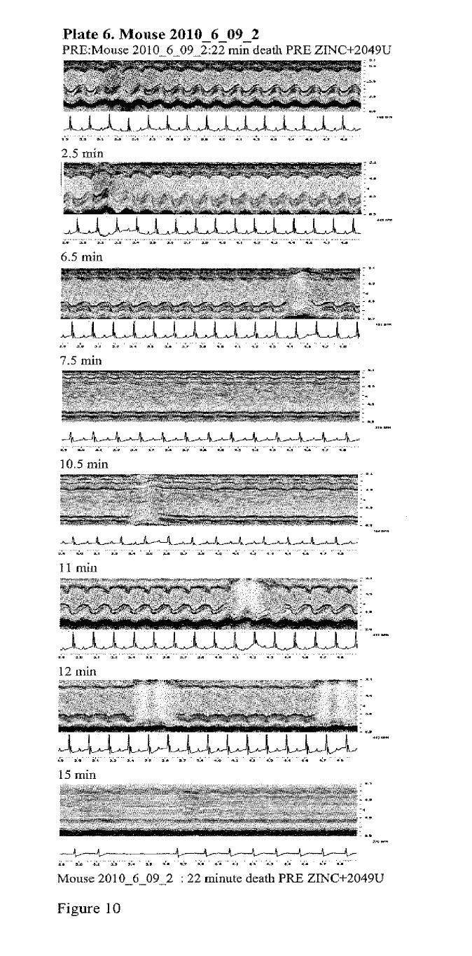

The mice pre-injected with zinc gluconate also exhibited profound decreases in ventricular contraction but rebounds were frequently observed subsequent to periods of pulseless electrical activity (PEA) in three of the zinc gluconate injected mice. For example, FIG. 10 (Mouse 2010_{—}6_{—}09_{—}2), illustrating representative data from zinc gluconate injected mice, shows a cessation of ventricular contraction ceased at 10.5 minutes, but recovered at 11 minutes. The EKG also recovered within thirty seconds.

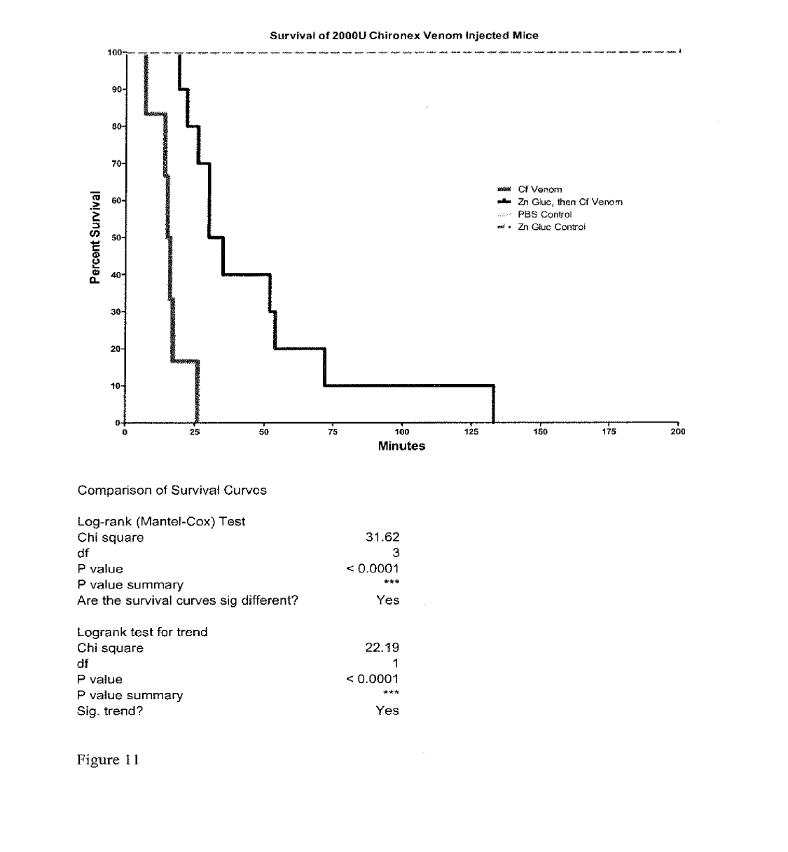

FIG. 11 is a compilation of the survival rate as well as duration of survival post-envenomation for untreated mice and zinc gluconate-treated mice. The results indicate that zinc gluconate-treated mice experienced higher survival rates as well as longer durations of survival relative to the untreated mice.

Table 3 illustrates survival times, plasma hemoglobin levels and potassium levels for exemplary mice in the study (both untreated and treated with zinc gluconate).

TABLE 3

| | | Survival | Plasma | Plasma | Hemol- |
|---|---|---|---|---|---|
| Animal | | Time | [K+] | Hgb | ysis |
| Number | Sample | (min) | mM | mg/100 mL | % |
| 2010-3-26-05 | Control PBS | NA | 5.1 | 150 | 1% |
| 2010-06-11-03 | 2000 U Crude | 2 | 38.0 | 6,058 | 40% |
| 2010-06-11-04 | 2000 U Crude | 16 | 11.5 | 516 | 3% |
| 2010-06-11-01 | 2000 U Crude | 26 | 9.1 | 875 | 6% |
| 2010-06-09-04 | Zn Gluc then | 4 | 16.4 | 860 | 6% |
| | 2000 U | | | | |
| 2010-06-09-05 | Zn Gluc then | 19 | 6.2 | 673 | 4% |
| | 2000 U | | | | |
| 2010-5-12-01 | Zn Gluc then | 72 | 7.5 | 348 | 2% |
| | 2000 U | | | | |
| 2010-06-09-02 | Zn Gluc then | 22 | 8.2 | 471 | 3% |
| | 2000 U | | | | |

### Conclusion

These studies of venom injected mice showed marked periods of PEA with EKG findings consistent with hyperkalemia. Plasma hemoglobin with potassium quantitation demonstrate that a catastrophic hyperkalemic state precedes clinically measurable hemolysis. ECHO of mice injected with purified hemolysin show identical responses to total crude venom shown here. Thus, the results indicate that these effects can be specifically attributed to the cubozoan PFTs.

The results also illustrate that zinc treated animals exposed to cubozoan PFT exhibited a significantly improved survival and maintained normal EKG conductive sequences for longer periods. Other improvements included the capacity for recovery. Survival times following Chironex fleckeri venom injection were significantly prolonged (P<0.0001) by the intravenous administration of zinc gluconate. These remarkable findings indicate the utility of the rapid administration of zinc gluconate in a subject exposed to Chironex fleckeri envenomation as well as to similar types of PFTs.

### EXAMPLE 7

A Zinc-Containing Compound Improves Cardiovascular Abnormalities in Piglets Exposed to A Pore-Forming Toxin

Experiments are conducted upon piglets exposed to a cubozoan porin to determine the physiological effects of a pore-forming toxin in vivo. The study is carried out to characterize cardiovascular hemodynamics, pulmonary function, organ perfusion, and whole body clearance of purified porin in ten anesthetized 8 kg pigs catheterized with arterial and venous lines for direct blood pressure measurement, blood sampling and administration of solutions and microspheres; and a bladder cannula for urine collection. Purified porin is injected into piglets at doses titrated to range from local inflammation to clinical Irukandji syndrome. Physiological responses are measured before and after injection of the porin. These include temperature, blood pressure, heart rate, cardiac output and electrocardiogram (EKG), pulmonary pressure volume loops, blood gasses, and renal output. Tissue microcirculatory perfusion and indices of hypoxemia and endocrine responses are also assessed.

### Protocols

Venom Preparation. Crude venom is isolated from freshly captured Hawaiian box jellyfish, Carybdea alata. Purified venom porin is isolated from the crude venom using multidimensional high pressure chromatography (HPLC) and other biochemical separation techniques as previously described (Chung et al. 2001. Toxicon 39:981-990, which is incorporated herein by reference in its entirety). The effects of crude or purified porin is examined on freshly drawn PBMCs or platelets prepared from platelet-enriched plasma. Specifically, dose-response time course incubations are performed with plasma isolated by end point centrifugation and snap freezing. Frozen cell-free plasma samples are then tested using cytokine panel assays or electrochemical detection based catecholamine assay.

In Vitro PBMC and Platelet Assays. Inflammatory cytokines, including PDGF, RANTES, MCP, G-CSF, TNF and TGF-beta are measured in the blood plasma using porcine antibody synthesized as described (Bjerre et al. 2009. Vet Immuno and Immunopath 130:53-58, which is incorporated herein by reference in its entirety) or commercially obtained (human with >75% porcine cross reactivity validated). Assays are carried out utilitzing a multiplex microsphere-bead based Immunoassay (MBIA) by the Bio-Plex array reader (Bio-Rad Laboratories, Hercules, Calif.) platform (Millipore, USA) with multiplex cytokine regents supplied by Luminex Inc (Austin, Texas, USA) on a Luminex-200™instrument using Exponent software (Invitrogen, Paisley, England).

### Piglet Studies

Ten 8 kg pigs are cardiac catheterized. Once catheterized, pigs are infused with a background infusion of normal saline i.v. at 0.1 ml/kg/min. Volume infusion is sustained throughout the experiment to maintain adequate hydration and central venous pressures as determined at baseline. Blood pressures and standard hemodynamic parameters are continuously monitored, and urine is collected throughout the experiment. After the initial 60-90 minute period of stabilization after catheterization, pigs are assessed for a 20 minute period defined as the baseline. Porin is slowly injected in increasing doses in 4 additional time periods for up to 60 minutes or until a steady-state is achieved. Zinc is administered according to routine neonatal intravenous administration protocols readily known to those of skill in the art.

Hemodynamic and Respiratory measurements. Hemodynamic parameters are monitored throughout the experiment. These include heart rate, blood pressure, central venous pressure, pulmonary wedge pressure via a Swan Ganz catheter, cardiac output by thermodilution, urine output, oxygen saturation, arterial blood gases, and respiratory rates. Systemic vascular resistance, pulmonary vascular resistance, and oxygen delivery and consumption are calculated from these direct measurements. Dynamic lung pressure volume curves are recorded for each period.

Assessment of microcirculatory blood flow shifts. Blood flow to individual organs including the brain, heart, kidney, liver, stomach, muscle and skin is calculated and compared to examine redistribution of flow within the body. To determine organ blood flows, different colored microspheres are injected into the systemic circulation at each of the periods for a total of 5 colors used. Organs harvested at the conclusion of the experiment is analyzed for microsphere distribution.

Blood sampling. Blood (approximately 7.0-8.0 mL per sample) is sampled for coagulation assessment, catecholamines, pro- and anti-inflammatory cytokines, catecholamines, vasopressin, cortisol, adrenal corticoptropic hormone (ACTH), aldosterone, lactate, and electrolytes. Plasma is separated, and red blood cells are returned in an equal volume of saline at the time of the next sampling to help maintain blood volume and oxygenation. Plasma for hormone analyses is measured by radioimmunoassay or ELISA. Arterial blood gases and mixed venous and arterial oxygen saturations are determined at each measurement session in addition to the blood samples for coagulation studies, hormone analyses, osmolality, and electrolyte analyses.

Urine sampling. Urine is collected continuously in preweighed tubes for gravimetric determination of urine volume and calculation of urine flow rate. Urine samples are analyzed for osmolality, creatinine (for estimation of GFR), and electrolytes.

Tissue sampling. Tissues from all vital organs are harvested at the end of the experiment, after euthanasia. Necropsy samples are obtained, including: heart, brain, liver, kidney, spleen, gut, lung, skin, and thigh muscle is harvested.

Data analysis. Assessments of cardiovascular, pulmonary, and endocrine function after each porin dose is compared with that at baseline via ANOVA with repeated measures over time. In addition the relationship between cytokine levels and catecholamine levels, and cardiorespiratory function indices are evaluated with multiple regression analysis. For all statistical tests, a p value<0.05 is considered statistically significant.

Administration of zinc gluconate is observed to siginificantly improve survival and reduce the severity of symptoms associated with exposure to the cubozoan porin.

### EXAMPLE 8

Use of A Zinc-Containing Compound to Treat A Human Subject Exposed to A Cubozoan Pore-Forming Toxin (Intravenous Administration)

In this Example, human subjects are treated with zinc gluconate intravenously after being envenomated by a cubozoan porin. Human subjects are quickly treated with intravenous infusion of 2.5 to 4 mg of zinc gluconate per day for several days. Samples are removed to monitor levels of potassium, cytokines, histamine, and catecholamine, in the blood serum. Administration of zinc gluconate is observed to reduce the severity of physiological symptoms associated with the venom poisoning and to decrease the likelihood a lethal outcome.

### EXAMPLE 9

Use of A Zinc-Containing Compound to Treat A Human Subject Exposed to A Cubozoan Pore-Forming Toxin (Subcutaneous Bolus Followed by Continuous IV Administration

In this Example, human subjects are treated with zinc gluconate by subcutaneous and intravenous means after being envenomated by a cubozoan porin. Human subjects are quickly injected subcutaneously with an initial zinc gluconate bolus (5 mL of a 100 mM solution appropriate for injection) followed by a continuous intravenous (IV) drop with 5 mM zinc gluconate at established rates of fluid infusion. Samples are taken to monitor levels of potassium, cytokines, histamine, and catecholamine, in the blood serum. Administration of zinc gluconate is observed to reduce the severity of physiological symptoms associated with the venom poisoning and to decrease the likelihood a lethal outcome.

### EXAMPLE 10

Use of A Zinc-Containing Compound to Treat A Human Subject Exposed to A Cubozoan Pore-Forming Toxin (Transdermal Administration

In this Example, human subjects are treated with zinc gluconate via a transdermal patch after being envenomated by a cubozoan porin. Human subjects afflicted by porin exposure are quickly administered with zinc gluconate by a transdermal patch using an iontophoresis unit set to deliver a 5 mM solution of zinc gluconate at 40 mAmp/min. Administration of zinc gluconate is observed to reduce the severity of physiological symptoms associated with the venom poisoning and to decrease the likelihood a lethal outcome.

### EXAMPLE 11

Use of A Zinc-Containing Compound to Treat A Human Subject Exposed to A Bacterial Pore-Forming Toxin (Intravenous Administration

In this Example, human subjects are treated with zinc gluconate intravenously after being exposed to a bacterial porin. Human subjects are treated with intravenous infusion of 2.5 to 4 mg of zinc gluconate per day for several days. Samples are removed to monitor levels of potassium, cytokines, histamine, and catecholamine, in the blood serum. Administration of zinc gluconate is observed to reduce the severity of physiological symptoms associated with porin exposure and to improve the health of the subject.

### EXAMPLE 12

Use of A Zinc-Containing Compound to Treat A Human Subject Exposed to A Viral Pore-Forming Toxin (Intravenous Administration

In this Example, human subjects are treated with zinc gluconate intravenously after being exposed to a viral porin. Human subjects are treated with an intravenous infusion of 2.5 to 4 mg of zinc gluconate per day for several days. Samples are removed to monitor levels of potassium, cytokines, histamine, and catecholamine, in the blood serum. Administration of zinc gluconate is observed to reduce the severity of physiological symptoms associated with porin exposure and to improve the health of the subject.

### EXAMPLE 13

Use of A Zinc-Containing Compound to Treat A Human Subject Exposed to A Mushroom Pore-Forming Toxin (Intravenous Administration

In this Example, human subjects are treated with zinc gluconate intravenously after being exposed to a mushroom pore-forming toxin. Human subjects are treated with an intravenous infusion of 2.5 to 4 mg of zinc gluconate per day for several days. Samples are removed to monitor levels of potassium, cytokines, histamine, and catecholamine, in the blood serum. Administration of zinc gluconate is observed to reduce the severity of physiological symptoms associated with porin exposure and to improve the health of the subject.

1. Use of a zinc-containing composition for the manufacture of a medicament for treating a disease or condition associated with a pore-forming toxin.

2. The use of claim 1, wherein the zinc containing compound is zinc gluconate.

3. The use of claim 1, wherein the disease or condition is selected from the group consisting of bacterial sepsis, Irukandji syndrome, cardiovascular collapse, pulseless electrical activity (PEA) hyperkalemia, hemolysis, cytokine and histamine release, and catecholamine surge.

4. The use of claim 1, wherein the composition additionally comprises a therapeutically effective dose of a carbohydrate.

5. The use of claim 4, wherein the carbohydrate comprises D-lactulose.

6. (canceled)

7. A method for treating a mammal suffering from a disease or condition resulting from the action of a pore-forming toxin, comprising administering to the mammal a therapeutically effective dosage of a zinc-containing compound.

8. The method of claim 7, wherein the zinc-containing compound is administered intravenously.

9. The method of claim 7, additionally comprising administering to the mammal a therapeutically effective dose of a composition comprising a carbohydrate.

10. A method for treating a mammal suffering from a disease resulting from the action of a pore-forming toxin, comprising administering to the mammal a therapeutically effective dosage of a composition comprising a carbohydrate.

11. The method of claim 10, wherein the carbohydrate is D-lactulose.
